(19) United States
(12) Patent Application Publication
(10) Pub. No.: US 2021/0121221 A1
(43) Pub. Date: Apr. 29, 2021

(54) TREATMENT TOOL

(71) Applicant: OLYMPUS CORPORATION, Tokyo (JP)

(72) Inventor: Masato NARISAWA, Tokyo (JP)

(73) Assignee: OLYMPUS CORPORATION, Tokyo (JP)

(21) Appl. No.: 17/143,337

(22) Filed: Jan. 7, 2021

Related U.S. Application Data
(63) Continuation of application No. PCT/JP2018/026402, filed on Jul. 12, 2018.

(51) Int. Cl. A61B 18/08

(52) U.S. Cl. CPC A61B 18/085; A61B 2018/00083

(57) ABSTRACT

A treatment tool includes: a treatment member configured to transmit heat to living tissue; an insulating substrate having a first surface that is joined to the treatment member and a second surface that is opposite to the first surface; a heat generation area that is arranged on the first surface, the heat generation area being configured to generate heat by supply of power; an interconnection area that is arranged on the second surface, the interconnection area being configured to supply the power to the heat generation area; and a first conductor configured to make conduction between the heat generation area and the interconnection area.

### CROSS-REFERENCE TO RELATED APPLICATION

This application is a continuation of International Application No. PCT/JP2018/026402, filed on Jul. 12, 2018, the entire contents of which are incorporated herein by reference.

### BACKGROUND

### 1. Technical Field

The disclosure relates to a treatment tool.

### 2. Related Art

A treatment tool that treats a region to be treated (subject region below) in living tissue by applying energy to the subject region has been known (for example, refer to Japanese Patent No. 5797348).

The treatment tool described in Japanese Patent No. 5797348 includes a pair of grip members that grip a subject region. In the grip member, a treatment member that makes contact with the subject region when the subject region is gripped with the pair of grip members and a heater for heating the treatment member are provided. In the treatment tool, heat from the heater is transmitted to the subject region that is gripped by the pair of grip members via the treatment member. The subject region is thus treated.

The heater described in Japanese Patent No. 5797348 includes a substrate and a resistive pattern that is formed on the substrate. The resistive pattern includes a heat generation area that generates heat by energization and first and second connection areas that are electrically connected to the heat generation areas and to which first and second wiring members that perform the energization are connected, respectively. The first and second connection areas are arranged in parallel in the direction of the width of the substrate at the side of the proximal end of the substrate. The heat generation area has an approximate U-shape in which the heat generation area extends from the proximal end side toward a distal end side on the substrate, turns at the distal end side, and extends toward the proximal end side. Both ends of the heat generation area have conduction with the first and second connection areas. In other words, the resistive pattern has two electric paths that are parallel in the width direction of the substrate.

### SUMMARY

In some embodiments, a treatment tool includes: a treatment member configured to transmit heat to living tissue; an insulating substrate having a first surface that is joined to the treatment member and a second surface that is opposite to the first surface; a heat generation area that is arranged on the first surface, the heat generation area being configured to generate heat by supply of power; an interconnection area that is arranged on the second surface, the interconnection area being configured to supply the power to the heat generation area; and a first conductor configured to make conduction between the heat generation area and the interconnection area.

In some embodiments, a treatment tool includes: a treatment member configured to transmit heat to living tissue; an insulating substrate having a first surface that is joined to the treatment member and a second surface that is opposite to the first surface; a first heat generation area that is formed on the first surface along a longitudinal direction of the substrate, the first heat generation area being configured to generate heat by supply of a first power; a second heat generation area that is formed on the first surface along the longitudinal direction and between the first heat generation area and a distal end of the substrate, the second heat generation area being configured to generate heat by supply of a second power; a first interconnection area that is formed in an intermediate layer of the substrate, the first interconnection area being configured to supply the first power to the first heat generation area; a second interconnection area that is formed in the intermediate layer of the substrate, the second interconnection area being configured to supply the second power to the second heat generation area; a third interconnection area that is formed on the second surface, the third interconnection area being configured to supply the second power to the second heat generation area; a first conductor configured to make conduction between the first heat generation area and the first interconnection area; a third conductor configured to make conduction between the second heat generation area and the second interconnection area; and a fourth conductor that is arranged more on a distal end side of the treatment member than the first conductor and the third conductor are and that enables conduction between the second heat generation area and the third interconnection area.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to a first embodiment;

FIG. 2 is an enlarged view of a distal end part of a treatment tool;

FIG. 3 is an exploded perspective view illustrating a heat generation structure;

FIG. 4 is a diagram illustrating a heater;

FIG. 5 is a diagram illustrating the heater;

FIG. 6 is a diagram illustrating the heater;

FIG. 7 is a diagram illustrating a heater according to a second embodiment;

FIG. 8 is a diagram illustrating the heater according to the second embodiment;

FIG. 9 is a diagram illustrating the heater according to the second embodiment;

FIG. 10 is a diagram illustrating the heater according to the second embodiment;

FIG. 11 is a diagram illustrating a heater according to a third embodiment;

FIG. 12 is a diagram illustrating the heater according to the third embodiment;

FIG. 13 is a diagram illustrating the heater according to the third embodiment; and

FIG. 14 is a diagram illustrating the heater according to the third embodiment.

### DETAILED DESCRIPTION

With reference to the accompanying drawings, modes for carrying out the disclosure (“embodiments” below) will be described below. The embodiments described below do not limit the disclosure. Furthermore, in the illustration of the drawings, the same components are denoted with the same reference number.

### First Embodiment

Schematic Configuration of Treatment System

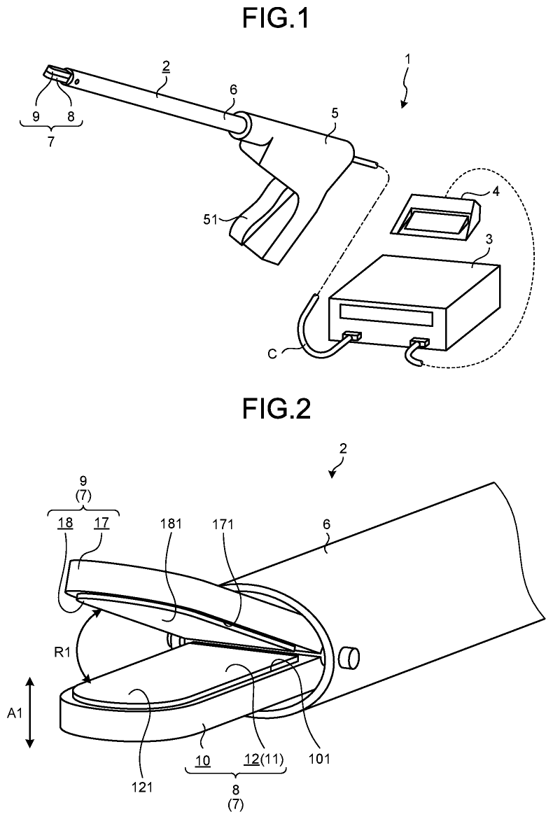

FIG. 1 is a diagram illustrating a treatment system 1 according to a first embodiment.

The treatment system 1 treats a region to be treated (“subject region”) in living tissue by applying thermal energy to the subject region. The treating herein refers to, for example, coagulating and cutting the subject region. As illustrated in FIG. 1, the treatment system 1 includes a treatment tool 2, a control device 3, and a footswitch 4.

Configuration of Treatment Tool

The treatment tool 2 is a surgical medical treatment tool for treating a subject region with the tool penetrating through the abdominal wall. As illustrated in FIG. 2, the treatment tool 2 includes a handle 5, a shaft 6, and a gripper 7.

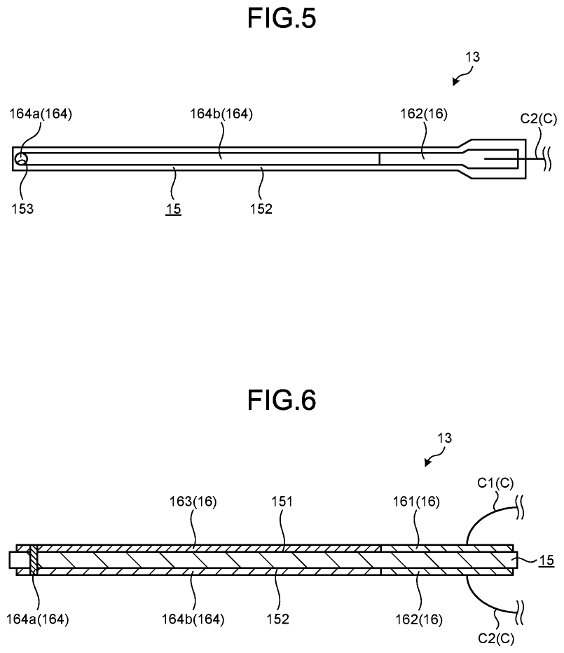

The handle 5 is a part that is held by a practitioner by hand. As illustrated in FIG. 5, an operation knob 51 is provided in the handle 5.

The shaft 6 is approximately cylindrical and one end of the shaft 6 is connected to the handle 5 (FIG. 1). The gripper 7 is connected to the other end of the shaft 6. An open-close mechanism (not illustrated in the drawing) that causes grip members 8 and 9 (FIG. 1) forming the gripper 7 to open or close in response to an operation by the practitioner on the operation knob 51 is provided in the shaft 6. An electric cable C (FIG. 1) is laid in the shaft 6 from one end side to the other end side via the handle 5.

Configuration of Gripper

The “distal end side” described below refers to the side of the distal end of the gripper 7 and means the left side in FIG. 1. The “proximal end side” described below refers to the side of the gripper 7 on the side of the shaft 6 and means the right side in FIG. 1.

FIG. 2 is an enlarged view of a distal end part of the treatment tool 2.

The gripper 7 is a part that treats a subject region while gripping the subject region. As illustrated in FIG. 1 or 2, the gripper 7 includes a first and second grip members 8 and 9.

The first and second grip members 8 and 9 are configured to be openable or closable in the direction of the arrow R1 (FIG. 2) in response to an operation of the practitioner on the operation knob 51.

Configuration of First Grip Member

The first grip member 8 is arranged on a lower side with respect to the second grip member 9 in FIG. 1 or FIG. 2. As illustrated in FIG. 2, the first grip member 8 includes a first jaw 10 and a heat generation structure 11.

The first jaw 10 is formed in an elongated shape extending in a longitudinal direction from the distal end of the gripper 7 to the proximal end (in the left-right direction in FIG. 1 and FIG. 2). In the first jaw 10, a concave 101 is formed on an upper surface.

The concave 101 is positioned at the center of the first jaw 10 in the width direction and extends along the longitudinal direction of the first jaw 10. Among side walls forming the concave 101, the side wall on the proximal end side is omitted.

The first jaw 10 is fixed to the end of the shaft 6 on the distal end side while supporting the heat generation structure 11 with the concave 101 in a posture in which the concave 101 faces up in FIG. 2.

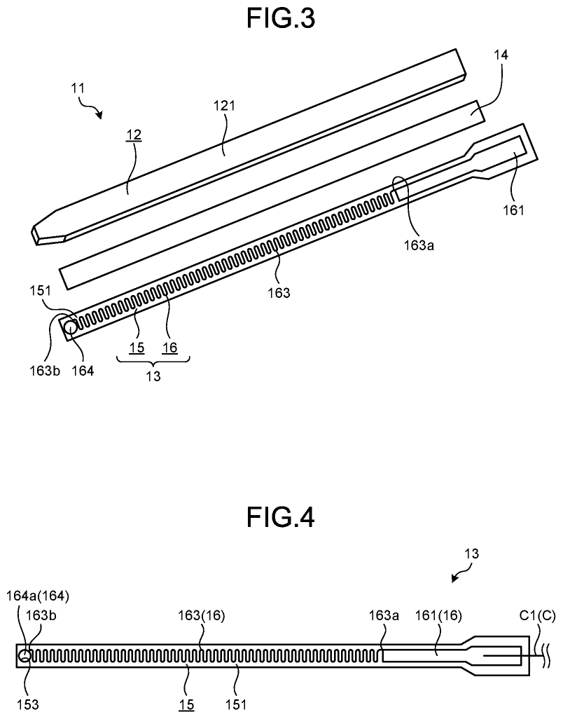

FIG. 3 is an exploded perspective view illustrating the heat generation structure 11. Specifically, FIG. 3 is an exploded perspective view of the heat generation structure 11 viewed from above in FIG. 1 and FIG. 2.

The heat generation structure 11 is housed in the concave 101 with part of the heat generation structure 11 protruding toward the upper side in FIG. 2. Under the control of the control device 3, the heat generation structure 11 generates thermal energy. As illustrated in FIG. 3, the heat generation structure 11 includes a heat transmission board 12, a heater 13, and an adherent member 14.

The heat transmission board 12 corresponds to a treatment member according to the disclosure. The heat transmission board 12 is an elongated board member that is formed of a material, such as copper, and that extends in a longitudinal direction of the gripper 7.

The heat transmission board 12 has an upper surface in FIGS. 2 and 3 that makes contact with the subject region in the state of being gripped by the first and second grip members 8 and 9. The surface transmits heat from the heater 13 to the subject region. In other words, the surface functions as a treatment surface 121 that applies thermal energy to the subject region (FIGS. 2 and 3). In the first embodiment, the treatment surface 121 is formed of a flat surface orthogonal to the direction A1 (FIG. 2) in which the first and second grip members 8 and 9 are faced each other while gripping the subject region.

In the first embodiment, the treatment surface 121 is formed of a flat surface. Alternatively, the treatment surface 121 may be formed of another shape, such as a convex shape or a concave shape. The same applies to a grip surface 181 to be described below.

FIGS. 4 to 6 are diagrams illustrating the heater 13. Specifically, FIG. 4 is a diagram of the heater 13 viewed from the side of the heat transmission board 12. FIG. 5 is a diagram of the heater 13 viewed from the side of the bottom surface of the concave 101. FIG. 6 is a cross-sectional view of the heater 13 taken along a plane orthogonal to the width direction of the heater 13 (the up-down direction in FIGS. 4 and 5).

The heater 13 is a sheet heater that partly generates heat and thus heats the heat transmission board 12. As illustrated in FIGS. 4 to 6, the heater 13 includes a substrate 15 and a resistive pattern 16.

The substrate 15 is an elongated sheet that is formed of an insulating material, such as polyimide, and that extends along the longitudinal direction of the gripper 7.

The material of the substrate 15 is not limited to polyimide, and a high heat resistance insulating material, such as aluminum nitride, alumina, glass, or zirconia, may be used.

According to the following description, the surface of the substrate 15 facing the heat transmission board 12 is referred to as a first surface 151 (FIGS. 4 and 6) and the surface opposite to the first surface 151 is referred to as a second surface 152 (FIG. 5 and FIG. 6).

The resistive pattern 16 is formed of a conductive material. As illustrated in FIGS. 4 to 6, the resistive pattern 16 includes a first connector 161 (FIGS. 4 and 6), a second connector 162 (FIGS. 5 and 6), a heat generator 163, and an interconnection unit 164.

The first connector 161 corresponds to a first connection area according to the disclosure. As illustrated in FIG. 4, the first connector 161 is formed on the first surface 151 and between the heat generator 163 and a proximal end of the substrate 15 (the right end in FIG. 4). A first lead line C1 that forms an electric cable C (FIGS. 4 and 6) is connected to the first connector 161. The first lad line C1 corresponds to a first wiring member according to the disclosure.

The second connector 162 corresponds to a second connection area according to the disclosure. As illustrated in FIG. 5, the second connector 162 is formed on the second surface 152 at a position across the substrate 15 from the first connector 161. A second lead line C2 that forms the electric cable C (FIGS. 5 and 6) is connected to the second connector 162. The second lead line C2 corresponds to a second wiring member according to the disclosure.

The heat generator 163 corresponds to a heat generation area according to the disclosure. As illustrated in FIG. 4, one end 163a of the heat generator 163 is positioned on the proximal end side on the first surface 151 and the heat generator 163 extends from the end 163a toward the distal end side in a zigzag. Another end 163b of the heat generator 163 is positioned on the distal end side on the first surface 151. The end 163a is electrically connected to the first connector 161. The end 163a corresponds to a first end according to the disclosure. The other end 163b corresponds to a second end according to the disclosure.

The interconnection unit 164 corresponds to an interconnection area according to the disclosure. As illustrated in FIGS. 4 to 6, the interconnection unit 164 includes a first conductor 164a and a second conductor 164b (FIGS. 5 and 6).

The first conductor 164a corresponds to a through hole internal area according to the disclosure.

As illustrated in FIGS. 4 to 6, a through hole 153 penetrating through the first and second surfaces 151 and 152 is formed at the distal-end side of the substrate 15.

The first conductor 164a is a conducting part that is formed in the through hole 153 and is electrically connected to the other end 163b of the heat generator 163. In other words, the first conductor 164a is a through hole. In the first embodiment, as illustrated in FIGS. 4 to 6, the first conductor 164a is embedded over the through hole 153.

A configuration in which the first conductor 164a is formed on the inner circumferential surface of the through hole 153 may be employed. The configuration in which the first conductor 164a is embedded as in the first embodiment increases the cross-sectional area and it is thus possible to lower the resistance value of the first conductor 164a compared to the configuration in which the first conductor 164a is provided only on the inner circumferential surface.

The second conductor 164b corresponds to a through hole external area according to the disclosure. As illustrated in FIG. 5 or 6, the second conductor 164b is provided on the second surface 152. One end of the second conductor 164b is electrically connected to the first conductor 164a, the second conductor 164b extends from the end toward the proximal end side, and the other end is electrically connected to the second connector 162. In the first embodiment, the width dimension of the second conductor 164b is larger than that of the heat generator 163 and is set at the same dimension as that of part of the second connector 162.

The resistance-temperature coefficient, resistance value, electrical resistivity of each of the first and second connectors 161 and 162 and the interconnection unit 164 are set smaller than those of the heat generator 163. The resistance-temperature coefficient, resistance value, electrical resistivity of the first conductor 164a are set smaller than those of the first and second connectors 161 and 162 and the second conductor 164b. As a material of which the heat generator 163 is formed, stainless steel, or the like, can be exemplified. As a material of which the first and second connectors 161 and 162 and the second conductor 164b are formed, gold, or the like, can be exemplified. For the first and second connectors 161 and 162 and the second conductor 164b, a configuration obtained by forming the first and second connectors 161 and 162 and the second conductor 164b using the same material as that of the heat generator 163 and then plating their surfaces with gold, or the like, may be employed. As a material of which the first conductor 164a is formed, copper, or the like, can be exemplified.

Under the control of the control device 3, a voltage is applied to the first and second connectors 161 and 162 via the first and second lead lines C1 and C2. Accordingly, in the resistive pattern 16, the heat generator 163 mainly generates heat.

As illustrated in FIG. 3, the adherent member 14 is an elongated sheet that is provided between the heat transmission board 12 and the first surface 151 of the substrate 15 and that extends along the longitudinal direction of the gripper 7. The adherent member 14 adheres to and fixes the heat transmission board 12 and the substrate 15. The adherent member 14 has preferable thermal conductivity and insulation property, is resistive to high temperatures, and is adherent.

As illustrated in FIG. 3, the heat transmission board 12 is arranged such that the heat transmission board 12 covers the heat generator 163. The adherent member 14 is arranged such that the adherent member 14 covers the whole heat generator 163 and covers part of the first connector 161. In other words, the adherent member 14 is arranged such that the adherent member 14 jets on the proximal end side with respect to the heat transmission board 12. The first lead line C1 is connected to an area that is not covered with the adherent member 14 in the first connector 161.

Configuration of Second Grip Member

As illustrated in FIG. 2, the second grip member 9 includes a second jaw 17 and an opposing board 18.

The second jaw 17 has the same shape as that of the first jaw 10. In other words, as illustrated in FIG. 2, the second jaw 17 has a concave 171 that is the same as the concave 101. The second jaw 17 is pivotally supported on the shaft 6 while supporting the opposing board 18 in the concave 171 such that the second jaw 17 is pivotable in a posture with the second jaw 17 facing down in FIG. 2 and, by pivoting, opens and closes with respect to the first grip member 8.

The first embodiment employs the configuration in which the first grip member 8 (the first jaw 10) is fixed to the shaft 6 and the second grip member 9 (the second jaw 17) is pivotally supported on the shaft 6; however, the configuration is not limited thereto. For example, a configuration in which both the first and second grip members 8 and 9 are pivotally supported on the shaft 6 and, by pivoting, each of the first and second grip members 8 and 9 opens or closes may be employed. For example, a configuration in which the first grip member 8 is pivotally supported on the shaft 6, the second grip member 9 is fixed to the shaft, and, by pivoting, the first grip member 8 opens and closes with respect to the second grip member 9 may be employed.

The opposing board 18 is formed of a conductive material, such as copper. The opposing board 18 is a flat board having a plane shape that is approximately the same as that of the concave 171 and is fixed in the concave 171. In the opposing board 18, the grip surface 181 on the lower side grips the subject region between the grip surface 181 and the treatment surface 121 in FIG. 2.

The opposing board 18 is not limited to a conductive material and the opposing board 18 may be formed using another material, such as polyether ether keton (PEEK).

Configuration of Control Device and Footswitch

The footswitch 4 is a part that is operated by the practitioner by foot. In response to the operation on the footswitch 4, the control device 3 executes treatment control.

The unit that cause the treatment control to be executed is not limited to the footswitch 4. Alternatively, a switch that is operated manually may be used.

The control device 3 includes a central processing unit (CPU), a field-programmable gate array (FPGA), or the like, and causes the treatment tool 2 to operate, thereby executing the treatment control to treat the subject region.

Operations of Treatment System

Operations of the above-described treatment system 1 will be described.

The practitioner holds the treatment tool 2 by hand and passes the distal end part of the treatment tool 2 (the gripper 7 and part of the shaft 6) through the abdominal wall using, for example, a trocar and inserts the distal end part into the abdominal wall. The practitioner operates the operation knob 51. The practitioner grips the subject region with the gripper 7. The practitioner then operates the footswitch 4. The control device 3 then executes the following treatment control.

The control device 3 applies a voltage to the first and second connectors 161 and 162 via the first and second lead lines C1 and C2. The control device 3 measures a resistance value of the resistive pattern 16 (“heater resistance” below) by, for example, a voltage drop method from the values of voltage and current applied to the resistive pattern 16. The control device 3 refers to resistance-temperature properties that are measured previously. The resistance-temperature properties are properties representing the relation between the heater resistance and the temperature of the heat generator 163 (heater temperature below). The control device 3 controls the heater resistance at a target resistance value corresponding to a target temperature in the resistance-temperature properties while varying the power supplied to the resistive pattern 16. This controls the heat generator 163 at the target temperature. In other words, the heat from the heat generator 163 that is controlled at the target temperature is transmitted to the subject region via the heat transmission board 12. The subject region is then cut while being coagulated.

According to the above-described first embodiment, the following effect is achieved.

In the treatment tool 2 according to the first embodiment, the end 163a of the heat generator 163 is electrically connected to the first connector 161 on the first surface 151 and the heat generator 163 extends from the end 163a toward the distal end side. The other and 163b of the heat generator 163 is electrically connected to the first conductor 164a that is a through hole. The first conductor 164a is electrically connected to the second connector 162 via the second conductor 164b that extends from the distal end side toward the proximal end side on the second surface 152.

In other words, the resistive pattern 16 includes the single electric path that is formed on the first surface 151 and the single electric path that is formed on the second surface 152. For this reason, it is not necessary to arrange two electric paths in parallel in the width direction of the substrate as in the conventional one, which makes it possible to reduce the width dimension of the substrate 15. The insulating substrate 15 is present between the single electric path formed on the first surface 151 and the single electric path formed on the second surface 152. This makes it possible to prevent occurrence of short in the resistive pattern 16.

In the treatment tool 2 according to the first embodiment, the resistance value and electrical resistivity of the interconnection unit 164 are set smaller than those of the heat generator 163.

This makes it possible to inhibit the interconnection unit 164 from generating heat during energization of the resistive pattern 16.

When the resistance-temperature coefficient of the interconnection unit 164 is large, the resistance value of the interconnection unit 164 tends to vary according to the temperature. In other words, there is a possibility that, due to the effect of heat generation of the heat generator 163 and the effect of release of heat because of a structure that makes contact with the interconnection unit 164, the heater temperature will vary from the target temperature and be at an unintended temperature.

In contrast, in the treatment tool 2 according to the first embodiment, the resistance-temperature coefficient of the interconnection unit 164 is set smaller than that of the heat generator 163. This makes it possible to reduce the above-described effect and accurately control the heater temperature at the target temperature.

The first conductor 164a is a through hole penetrating through the first and second surfaces 151 and 152. For this reason, forming the first conductor 164a reduces the area in which the heat generator 163 is formed by the area in which the first conductor 164a is formed. In other words, in order to increase the area in which the heat generator 163 is formed as much as possible, it is preferable that the size of the first conductor 164a be small. On the other hand, reducing the size of the first conductor 164a increases the resistance value of the first conductor 164a and thus there is a risk of local excessive heating and breaking.

To deal with this, in the treatment tool 2 according to the first embodiment, the resistance-temperature coefficient, resistance value, and electrical resistivity of the first conductor 164a are set smaller than those of the second conductor 164b, respectively. This makes it possible to, even when the size of the first conductor 164a is reduced, inhibit local excessive heating and reduce the risk of breaking.

### Second Embodiment

A second embodiment will be described.

In the following description, the same components and steps as those of the first embodiment described above are denoted with the same reference numbers and detailed description thereof will be omitted or simplified.

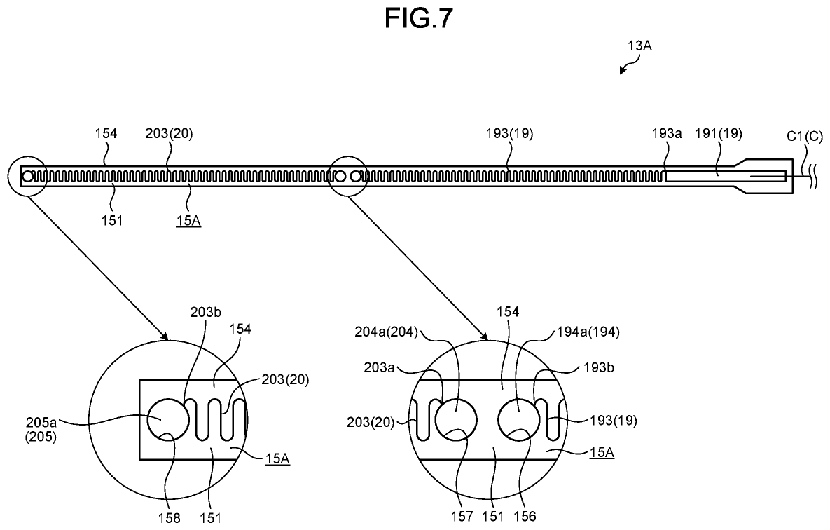

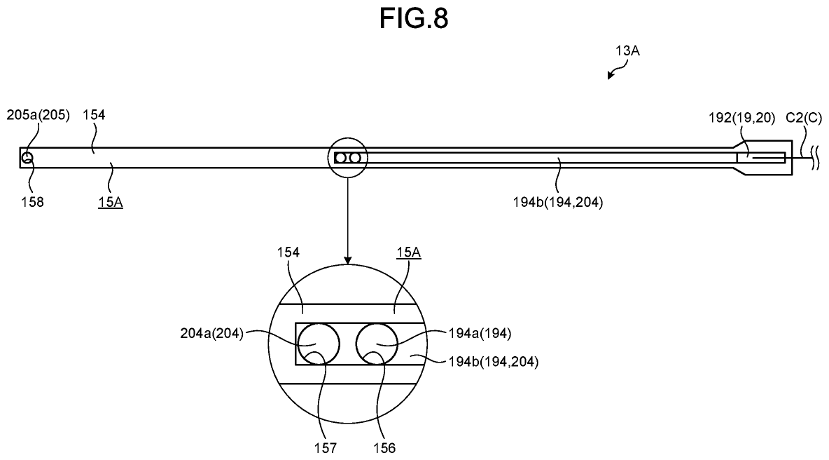

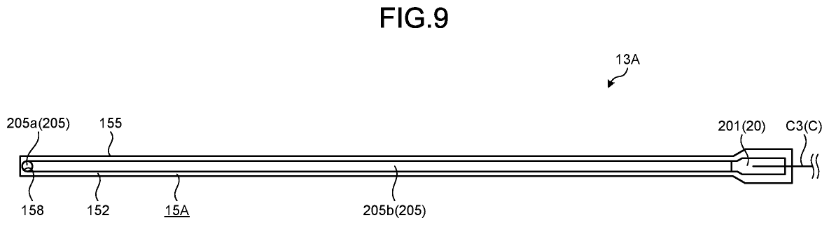

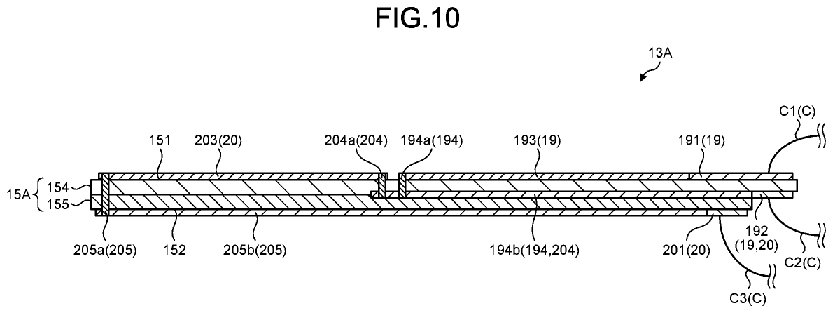

FIGS. 7 to 10 are diagrams illustrating a heater 13A according to the second embodiment. Specifically, FIG. 7 is a diagram of a first layer of the heater 13A viewed from the side of the heat transmission board 12. FIG. 8 is a diagram of a second layer of the heater 13A viewed from the side of the bottom surface of the concave 101. FIG. 9 is a diagram of a third layer of the heater 13A viewed from the side of the bottom surface of the concave 101. FIG. 10 is a cross-sectional view of the heater 13A taken along a plane orthogonal to the width direction of the heater 13A.

In the second embodiment, as illustrated in FIGS. 7 to 10, compared to the above-described first embodiment, the heater 13A is used instead of the heater 13.

The heater 13A is formed of a multilayer board. As illustrated in FIGS. 7 to 10, the heater 13A includes a substrate 15A, a first resistive pattern 19 (FIGS. 7, 8 and 10) and a second resistive pattern 20.

The substrate 15A is formed by laminating a first substrate layer 154 and a second substrate layer 155. Each of the first and second substrate layers 154 and 155 has approximately the same plane shape as that of the substrate 15 described in the above-described first embodiment and is formed of the same material as that of the substrate 15. The first substrate layer 154 is faced the heat transmission board 12 and has the first surface 151. On the other hand, the second substrate layer 155 is faced the bottom surface of the concave 101 and has the second surface 152. In the second embodiment, the length dimension of the first substrate layer 154 in the longitudinal direction is set larger than that of the second substrate layer 155. The first and second substrate layers 154 and 155 are laminated with the distal end side of the first substrate layer 154 jetting to the proximal end side compared to the second substrate layer 155. In the heater 13A that is a multi-layer board, a first layer is a wiring pattern that is formed on the first surface 151. A third layer is a wiring pattern that is formed on the second surface 152. A second layer is a wiring pattern that is formed on an intermediate layer of the substrate 15A, that is, on an interfacial face between the first substrate layer 154 and the second substrate layer 155.

The first resistive pattern 19 is formed of a conductive material. As illustrated in FIG. 7, 8 or 10, the first resistive pattern 19 includes a first connector 191 (FIGS. 7 and 10), a second connector 192 (FIGS. 8 and 10), a first heat generator 193 (FIGS. 7 and 10), and a first interconnection unit 194.

The first connector 191 corresponds to the first connection area according to the disclosure. As illustrated in FIG. 7 or FIG. 10, the first connector 191 is formed on the first surface 151 and between the first heat generator 193 and a proximal end of the substrate 15A, thereby forming the first layer of the heater 13A. The first lead line forming the electric cable C (FIGS. 7 and 10) is connected to the first connector 191. The first lead line C1 corresponds to the first wiring member according to the disclosure.

The second connector 192 corresponds to the second connection area according to the disclosure. As illustrated in FIG. 8 or FIG. 10, the second connector 192 is formed on the intermediate layer of the substrate 15A at a position facing the first connector 191, thereby forming the second layer of the heater 13A. The second connector 192 is exposed to the outside of the heater 13A. The second lead line C2 (FIGS. 8 and 10) forming the electric cable C is connected to the second connector 192. The second lead line C2 corresponds to the second wiring member according to the disclosure.

The first heat generator 193 corresponds to a first heat generation area according to the disclosure. As illustrated in FIG. 7, one end 193a of the first heat generator 193 is positioned on the proximal end side on the first surface 151 and the first heat generator 193 extends from the end 193a toward the distal end side in a zigzag. Another end 193b of the first heat generator 193 is positioned near the approximate center on the first surface 151 in the longitudinal direction of the substrate 15A. The end 193a is electrically connected to the first connector 191. The end 193a corresponds to the first end according to the disclosure. The other end 193b corresponds to the second end according to the disclosure.

The first interconnection unit 194 corresponds to a first interconnection area according to the disclosure. As illustrated in FIG. 7, 8 or 10, the first interconnection unit 194 includes a first conductor 194a and a second conductor 194b (FIGS. 8 and 10).

The first conductor 194a corresponds to a first hole internal area according to the disclosure.

In the substrate 15A, approximately at the center part on the substrate 15A in the longitudinal direction, as illustrated in FIG. 7, 8 or 10, first and second holes 156 and 157 each penetrating through the front and back of the first substrate layer 154 and extending from the first surface 151 to the intermediate layer of the substrate 15A are formed. The first hole 156 is positioned more on the proximal end side than the second hole 157 is. At the distal end side of the substrate 15A, as illustrated in FIGS. 7 to 10, a through hole 158 penetrating through the first and second surfaces 151 and 152 is formed. In other words, each of the first and second holes 156 and 157 is positioned more on the proximal end side than the through hole 158 is.

The first conductor 194a is a conducting part that is provided in the first hole 156 and is electrically connected to the other end 193b of the first heat generator 193. In other words, the first conductor 194a is a through hole. In the second embodiment, as illustrated in FIG. 7, 8 or 10, the first conductor 194a is embedded over the first hole 156.

The first conductor 194a may be configured such that the first conductor 194a is formed on only the inner circumferential surface of the first hole 156. In the configuration in which the first conductor 194a is embedded as in the second embodiment, the cross-sectional area increases and this makes is possible to lower the resistance value of the first conductor 194a compared to the configuration in which the first conductor 194a is provided only on the inner circumferential surface.

The second conductor 194b corresponds to a first hole external area according to the disclosure. As illustrated in FIG. 8 or 10, the second conductor 194b is provided on the intermediate layer of the substrate 15A and forms a second layer of the heater 13A. One end of the second conductor 194b is electrically connected to the first conductor 194a, the second conductor 194b extends from the end to the proximal end side, and the other end is electrically connected to the second connector 192. In the second embodiment, the width dimension of the second conductor 194b is larger than that of the first heat generator 193 and is set at the same dimension as that of the second connector 192.

The resistance-temperature coefficient, resistance value, electrical resistivity of each of the first and second connectors 191 and 192 and the first interconnection unit 194 are set smaller than those of the first heat generator 193. The resistance-temperature coefficient, resistance value, electrical resistivity of the first conductor 194a are set smaller than those of the first and second connectors 191 and 192 and the second conductor 194b. As a material of which the first heat generator 193 is formed, stainless steel, or the like, can be exemplified. As a material of which the first and second connectors 191 and 192 and the second conductor 194b are formed, gold, or the like, can be exemplified. For the first and second connectors 191 and 192 and the second conductor 194b, a configuration obtained by forming the first and second connectors 191 and 192 and the second conductor 194b using the same material as that of the first heat generator 193 and then plating their surfaces with gold, or the like, may be used. As a material of which the first conductor 194a is formed, copper, or the like, can be exemplified.

Under the control of the control device 3, voltage (corresponding to a first power according to the disclosure) is applied to the first and second connectors 191 and 192 via the first and second lead lines C1 and C2. Accordingly, in the first resistive pattern 19, the first heat generator 193 mainly generates heat.

The second resistive pattern 20 is formed of a conductive material. As illustrated in FIGS. 7 to 10, the second resistive pattern 20 includes the second connector 192 (FIGS. 8 and 10), a third connector 201 (FIG. 9 and FIG. 10), a second heat generator 203 (FIGS. 7 and 10), a second interconnection unit 204 (FIGS. 7, 8 and 10), and a third interconnection unit 205 (FIGS. 8 and 10).

The third connector 201 corresponds to a fourth connection area according to the disclosure. As illustrated in FIG. 9 or FIG. 10, the third connector 201 is formed on the second surface 152 at a position across the substrate 15A from the first connector 191, thereby forming the third layer of the heater 13A. A third lead line C3 forming the electric cable C (FIGS. 9 and 10) is connected to the third connector 201. The third lead line C3 corresponds to a fourth wiring member according to the disclosure.

The second heat generator 203 corresponds to a second heat generation area according to the disclosure. As illustrated in FIG. 7, the second heat generator 203 is formed on the first surface 151 and between the first heat generator 193 and the distal end of the substrate 15A. More specifically, one end 203a of the second heat generator 203 is positioned at an approximate center part in the longitudinal direction of the substrate 15A and the second heat generator 203 extends from the end 203a toward the distal end side in a zigzag. Another end 203b of the second heat generator 203 is positioned at the distal end side of the substrate 15A. The end 203a corresponds to a third end according to the disclosure. The other end 203b corresponds to a fourth end according to the disclosure.

As described above, the first and second heat generators 193 and 203 are arranged in different positions in the longitudinal direction of the substrate 15A.

The second interconnection unit 204 corresponds to a second interconnection area. As illustrated in FIG. 7, 8 or 10, the second interconnection unit 204 includes a second conductor 194b (FIGS. 8 and 10) and a third conductor 204a.

The third conductor 204a corresponds to a second hole internal area according to the disclosure. The third conductor 204a is a conductive part that is provided in the second hole 157 and electrically connects the end 203a of the second heat generator 203 and the second conductor 194b to each other. In other words, the third conductor 204a is a through hole. In the second embodiment, the third conductor 204a is embedded over the second hole 157 as illustrated in FIG. 7, 8 or 10.

A configuration in which the third conductor 204a is formed only on the inner circumferential surface of the second hole 157 may be employed. The configuration in which the third conductor 204a is embedded as in the second embodiment increases the cross-sectional area and it is thus possible to lower the resistance value of the third conductor 204a compared to the configuration in which the third conductor 204a is formed only on the inner circumferential surface.

As described above, the second heat generator 203 is electrically connected to the second connector 192 via the third conductor 204a and the second conductor 194b. In other words, the first and second resistive patterns 19 and 20 shares the second conductor 194b and the second connector 192. The second conductor 194b also has a function serving as a second hole external area according to the disclosure. The second connector 192 also has a function serving as a third connection area according to the disclosure. The second lead line C2 that is connected to the second connector 192 also has a function serving as a third wiring member according to the disclosure.

The third interconnection unit 205 corresponds to a third interconnection area according to the disclosure. As illustrated in FIGS. 7 to 10, the third interconnection unit 205 includes a fourth conductor 205a and a fifth conductor 205b (FIGS. 9 and 10).

The fourth conductor 205a correspond to a hole internal area according to the disclosure. The fourth conductor 205a is a conductive part that is provided in the through hole 158 and is electrically connected to the other end 203b of the second heat generator 203. In other words, the fourth conductor 205a is a through hole. In the second embodiment, the fourth conductor 205a is embedded over the through hole 158 as illustrated in FIGS. 7 to 10.

A configuration in which the fourth conductor 205a is formed only on the inner circumferential surface of the through hole 158 may be employed. The configuration in which the fourth conductor 205a is embedded as in the second embodiment increases the cross-sectional area and it is thus possible to lower the resistance value of the fourth conductor 205a compared to the configuration in which the fourth conductor 205a is formed only on the inner circumferential surface.

The fifth conductor 205b corresponds to the through hole external area according to the disclosure. As illustrated in FIG. 9 or 10, the fifth conductor 205b is provided on the second surface 152, thereby forming the third layer of the heater 13A. One end of the fifth conductor 205b is electrically connected to the fourth conductor 205a, the fifth conductor 205b extends from the end toward the proximal end side, and the other end is electrically connected to the third connector 201. In the second embodiment, the width dimension of the fifth conductor 205b is larger than that of the second heat generator 203 and is set at the same width dimension as part of the third connector 201.

The resistance-temperature coefficient, resistance value, electrical resistivity of each of the second and third connectors 192 and 201 and the interconnection units 204 and 205 are set smaller than those of the second heat generator 203. The resistance-temperature coefficient, resistance value, electrical resistivity of each of the third and fourth conductors 204a and 205a are set smaller than those of the second and third connectors 192 and 201 and the second and fifth conductors 194b and 205b. As a material of which the second heat generator 203 is formed, stainless steel, or the like, can be exemplified. As a material of which the third connector 201 and the fifth conductor 205b are formed, gold, or the like, can be exemplified. For the third connector 201 and the fifth conductor 205b, a configuration obtained by forming the third connector 201 and the fifth conductor 205b using the same material as that of the second heat generator 203 and then plating their surfaces with gold, or the like, may be employed. As a material of which the third and fourth conductors 204a and 205a are formed, copper, or the like, can be exemplified.

Under the control of the control device 3, a voltage (corresponding to a second power according to the disclosure) is applied to the second and third connectors 192 and 201 via the second and third lead lines C2 and C3. Accordingly, in the second resistive pattern 20, the second heat generator 203 mainly generates heat.

In the second embodiment, the control device 3 executes the following processing control.

Specifically, the control device 3 executes processing control to switch between a first state and a second state at given control intervals. The first state is a state in which a voltage is applied to the first and second connectors 191 and 192 via the first and second lead lines C1 and C2. In other word, the first state is a state in which only the first resistive pattern 19 among the first and second resistive patterns 19 and 20 is energized. In the first state, the control device 3 maintains a high potential in the first connector 191 and maintains a low potential (for example, a ground potential) in the second connector 192. The second state is a state in which a voltage is applied to the second and third connectors 192 and 201 via the second and third lead lines C2 and C3. In other words, the second state is a state in which only through the second resistive pattern 20 among the first and second resistive patterns 19 and 20 is energized. In the second state, the control device 3 maintains a high potential in third connector 201 and maintains a low potential in the second connector 192 (for example, a ground potential).

During execution of the treatment control, the control device 3 measures first and second heater resistances by, for example, the voltage drop method from the values of voltage and current applied to the first resistive pattern 19 or the second resistive pattern 20. The first heater resistance means the resistance value of the first resistive pattern 19. The second heater resistance means the resistance value of the second resistive pattern 20. The control device 3 refers to first and second resistance-temperature properties that are measured previously. The first resistance-temperature properties are properties representing the relation between the first heater resistance and the temperature of the first heat generator 193 (“first heater temperature” below). The second resistance-temperature properties are properties representing the relation between the second heater resistance and the temperature of the second heat generator 203 (“second heater temperature” below). The control device 3 controls the first and second heater resistances at target resistance values corresponding to target temperatures in the first and second resistance-temperature properties while varying the power supplied to the first and second resistive patterns 19 and 20. This controls the first and second heat generators 193 and 203 at the target temperatures independently. In other words, heat from the first and second heat generators 193 and 203 that are controlled at the target temperatures is transmitted to the subject region via the heat transmission board 12. The subject region is cut while being coagulated.

According to the above-described second embodiment, in addition to achieving the same effect as that of the above-described first embodiment, it is possible to solve the problem of uneven load.

The uneven load herein means the state in which the subject region is gripped not by the whole treatment surface 121 but by part of the treatment surface 121.

As in the above-described first embodiment, when the single heat generator 163 is provided over the area overlapping the treatment surface 121 in the thickness direction A1 of the substrate 15 (“treatment area” below), there is a risk that the following problem will occur.

When an uneven load occurs, in the heat generator 163, the temperature of a part that is covered with the subject region is lower than the target temperature because heat is transmitted to the subject region. On the other hand, in the heat generator 163, the temperature of the part not covered with the subject region is higher than the target temperature because heat is not transmitted to the subject region. In other words, it is not possible to heat the subject region at the target temperature and thus there is a risk that the time of treatment will take long.

To deal with this, in the heater 13A according to the second embodiment, the first and second heat generators 193 and 203 are provided in different positions in the longitudinal direction of the gripper 7. The first and second heat generators 193 and 203 are controlled independently. Thus, even during the uneven load, it is possible to heat the subject region at the target temperature and treat the subject region appropriately.

### Third Embodiment

A third embodiment will be described.

In the following description, the same components and steps as those of the first and second embodiments described above are denoted with the same reference numbers and detailed description thereof will be omitted or simplified.

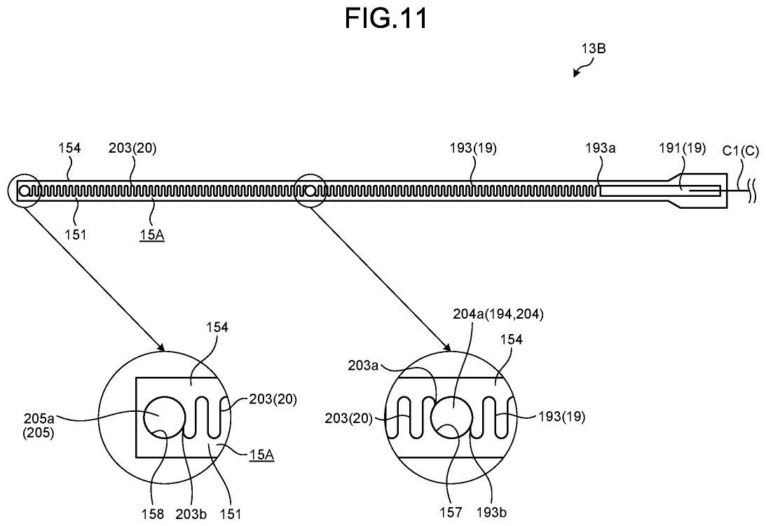

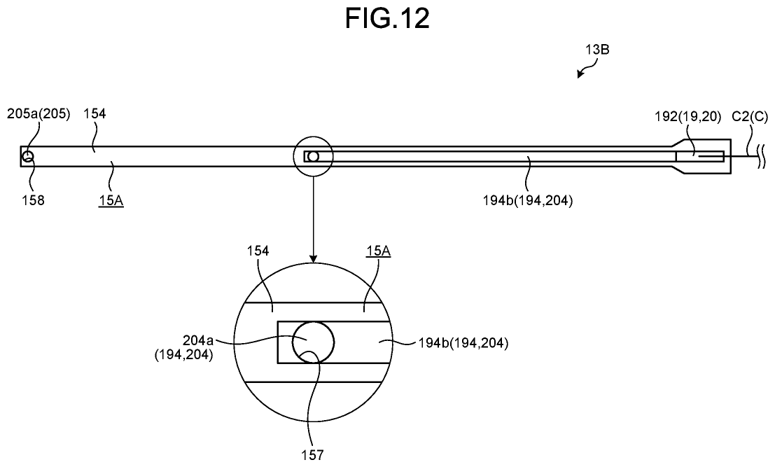

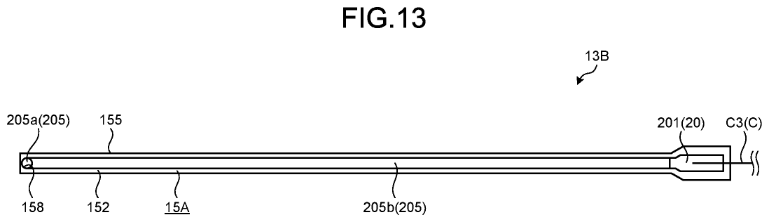

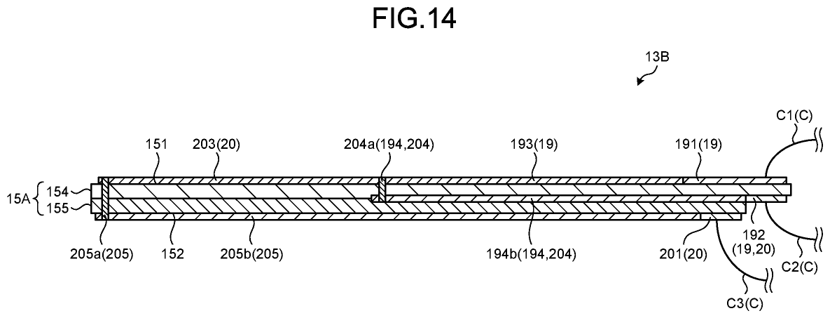

FIGS. 11 to 14 are diagrams illustrating a heater 13B according to the third embodiment. Specifically, FIG. 11 is a diagram corresponding to FIG. 7. FIG. 12 is a diagram corresponding to FIG. 8. FIG. 13 is a diagram corresponding to FIG. 9. FIG. 14 is a diagram corresponding to FIG. 10.

In the third embodiment, as illustrated in FIGS. 11 to 14, compared to the above-described first embodiment, the heater 13B is employed instead of the heater 13.

In the heater 13B, as illustrated in FIGS. 11 to 14, compared to the heater 13A described in the above-described second embodiment, the first interconnection unit 194 and the second interconnection unit 204 are formed as a common single interconnection area. Specifically, in the heater 13B, the first hole 156 and the first conductor 194a are not provided. The other end 193b of the first heat generator 193 is electrically connected to the third conductor 204a.

In other words, the first resistive pattern 19 includes the first and second connectors 191 and 192, the first heat generator 193, and the first interconnection unit 194 including the second and third conductors 194b and 204a. On the other hands, the second resistive pattern 20 includes the second and third connectors 192 and 201, the second heat generator 203, the second interconnection unit 204 including the second and third conductors 194b and 204a, and the third interconnection unit 205 including the fourth and fifth conductors 205a and 205b. The second hole 157 also has a function serving as the first hole according to the disclosure. The third conductor 204a also has the function serving as the first hole internal area according to the disclosure.

According to the third embodiment described above, the following effect is achieved in addition to the same effects as those of the above-described first and second embodiments.

In the heater 13B according to the third embodiment, the first and third conductors 194a and 204a are formed as the common single third conductor 204a. This reduces the area of the through hole, which makes it possible to increase the area in which the first and second heat generators 193 and 203 are formed and increase the heat generation area.

### Other Embodiments

The modes for carrying out the disclosure have been described, and the disclosure should not be limited to only the above-described first to third embodiments.

A configuration in which the heater 13, 13A or 13B is arranged in the above-described first to third embodiments and thermal energy is applied to a subject region from both the first and second grip members 8 and 9 may be employed.

A configuration in which, in addition to thermal energy, high-frequency energy or ultrasonic energy may be further applied to the subject region in the above-described first to third embodiments may be employed. Note that “applying high-frequency energy to the subject region” means causing a high-frequency current to be flown to the subject region and “applying ultrasonic energy to the subject region” means applying ultrasound vibrations to the subject region.

In the above-described second and third embodiments, only two resistive patterns that are the first and second resistive patterns 19 and 20 are provided. Alternatively, three or more resistive patterns may be provided. In this case, three or more heat generators including the first and second heat generators 193 and 203 are provided in different positions in the longitudinal direction of the substrate 15A.

In the above-described second and third embodiments, the substrate 15A includes two substrate layers that are the first and second substrate layers 154 and 155. Alternatively, the substrate 15A may be formed of at least three substrate layers.

In the above-described second embodiment, the first and second hole external areas according to the disclosure are formed as the common single second conductor 194b and the second and third connection areas according to the disclosure are formed as the common single second connector 192. Alternatively, the first and second hole external areas and the second and third connection areas may be provided independently. When the substrate according to the disclosure includes at least three substrate layers, the set of the first hole external area and the second connection area according to the disclosure and the set of the second hole external area and the third connection area according to the disclosure may be formed respectively on different intermediate layers among the intermediate layers. In this case, the second wiring member (the second lead line C2) according to the disclosure is connected to the second connection area and the third wiring member according to the disclosure different from the second lead line C2 is connected to the third connection area.

According to the treatment tool according to the disclosure, it is possible to reduce the width dimension of the substrate and prevent a short in the resistive pattern that is provided on the substrate.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a treatment member configured to transmit heat to living tissue;
an insulating substrate having a first surface that is joined to the treatment member and a second surface that is opposite to the first surface;
a heat generation area that is arranged on the first surface, the heat generation area being configured to generate heat by supply of power;
an interconnection area that is arranged on the second surface, the interconnection area being configured to supply the power to the heat generation area; and
a first conductor configured to make conduction between the heat generation area and the interconnection area.

2. The treatment tool according to claim 1, wherein the interconnection area has a resistance-temperature coefficient smaller than a resistance-temperature coefficient of the heat generation area.

3. The treatment tool according to claim 1, wherein the interconnection area has a resistance value smaller than a resistance value of the heat generation area.

4. The treatment tool according to claim 3, wherein the interconnection area has an electrical resistivity smaller than an electrical resistivity of the heat generation area.

5. The treatment tool according to claim 1, wherein the interconnection area has a resistance-temperature coefficient, a resistance value, and an electrical resistivity that are smaller than a resistance-temperature coefficient, a resistance value, and an electrical resistivity of the heat generation area, respectively.

6. The treatment tool according to claim 1, wherein the first conductor has a resistance-temperature coefficient smaller than a resistance-temperature coefficient of the interconnection area.

7. The treatment tool according to claim 1, wherein the first conductor has a resistance value smaller than a resistance value of the interconnection area.

8. The treatment tool according to claim 7, wherein the first conductor has an electrical resistivity smaller than an electrical resistivity of the interconnection area.

9. The treatment tool according to claim 1, wherein the first conductor has a resistance-temperature coefficient, a resistance value, and an electrical resistivity that are smaller than a resistance-temperature coefficient, a resistance value, and an electrical resistivity of the interconnection area, respectively.

10. The treatment tool according to claim 1, wherein the first conductor is a through hole configured to penetrate through the first surface and the second surface.

11. The treatment tool according to claim 1, wherein
the heat generation area extends along a longitudinal direction of the substrate,
the treatment tool further comprises:
a first connection area that is formed on the first surface and between the heat generation area and a proximal end of the substrate, that is electrically connected to a first end of the heat generation area in the longitudinal direction and to which a first wiring member that supplies the power is connected; and
a second connection area that is formed on the second surface at a position across the substrate from the first connection area and to which a second wiring member that supplies the power is connected, and
the first conductor electrically is configured to connect a second end of the heat generation area in the longitudinal direction and the second connection area.

12. A treatment tool comprising:
a treatment member configured to transmit heat to living tissue;
an insulating substrate having a first surface that is joined to the treatment member and a second surface that is opposite to the first surface;
a first heat generation area that is formed on the first surface along a longitudinal direction of the substrate, the first heat generation area being configured to generate heat by supply of a first power;
a second heat generation area that is formed on the first surface along the longitudinal direction and between the first heat generation area and a distal end of the substrate, the second heat generation area being configured to generate heat by supply of a second power;
a first interconnection area that is formed in an intermediate layer of the substrate, the first interconnection area being configured to supply the first power to the first heat generation area;
a second interconnection area that is formed in the intermediate layer of the substrate, the second interconnection area being configured to supply the second power to the second heat generation area;
a third interconnection area that is formed on the second surface, the third interconnection area being configured to supply the second power to the second heat generation area;
a first conductor configured to make conduction between the first heat generation area and the first interconnection area;
a third conductor configured to make conduction between the second heat generation area and the second interconnection area; and
a fourth conductor that is arranged more on a distal end side of the treatment member than the first conductor and the third conductor are and that enables conduction between the second heat generation area and the third interconnection area.

13. The treatment tool according to claim 12, wherein
the first conductor is a first hole configured to penetrate through the first surface and the intermediate layer of the substrate;
the third conductor is a second hole configured to penetrate through the first surface and the intermediate layer of the substrate; and
the fourth conductor is a through hole configured to penetrate through the first surface and the second surface.

14. The treatment tool according to claim 13, further comprising:
a first connection area that is formed on the first surface and between the first heat generation area and a proximal end of the substrate, that is electrically connected to a first end of the heat generation area in the longitudinal direction and to which a first wiring member that supplies the first power is connected; and
a second connection area that is formed on the intermediate layer of the substrate at a position across a first substrate layer substrate from the first connection area and to which a second wiring member that supplies the first power is connected, and
the first interconnection area is configured to electrically connect a second end of the first heat generation area in the longitudinal direction and the second connection area.

15. The treatment tool according to claim 14, further comprising:
a third connection area that is formed on the intermediate layer of the substrate at a position across the first substrate layer substrate from the first connection area and to which a third wiring member that supplies the second power is connected; and
a fourth connection area that is formed on the second surface at a position across the substrate from the first connection area and to which a fourth wiring member that supplies the second power is connected, wherein
the second interconnection area is configured to electrically connect a third end of the second heat generation area in the longitudinal direction and the third connection area, and
the third interconnection area is configured to electrically connect a fourth end of the second heat generation area in the longitudinal direction and the fourth connection area.

16. The treatment tool according to claim 15, wherein each of the first hole and the second hole extends from the first surface to the intermediate layer of the substrate,
the first interconnection area includes
a first hole internal area that is formed in the first hole and that is electrically connected to the second end; and
a first hole external area that is formed in the intermediate layer of the substrate, the first hole external area being configured to electrically connect the first hole internal area and the second connection area,
the second interconnection area includes
a second hole internal area that is formed in the second hole and that is electrically connected to the third end; and
a second hole external area that is formed in the intermediate layer of the substrate, the second hole external area being configured to electrically connect the second hole internal area and the third connection area, and
the third interconnection area includes
a through hole internal area that is formed in the through hole and that is electrically connected to the fourth end; and
a through hole external area that is formed on the second surface, the through hole external area being configured to electrically connect the trough hole internal area and the fourth connection area.

17. The treatment tool according to claim 16, wherein the first hole and the second hole are a shared hole;
the first hole internal area and the second hole internal area are a shared area,
the first hole external area and the second hole external area are a shared area, and
the second connection area and the third connection area are a shared area.
